(19) United States
(12) Patent Application Publication
(10) Pub. No.: US 2003/0226194 A1
(43) Pub. Date: Dec. 11, 2003

(54) Hair restraint device for masks and goggles

(76) Inventor: Alan Vossler, Baytown, TX (US)

Correspondence Address: Stephen Schlather, 3406 Harbour Breeze Lane, Pearland, TX, 77584, US

(21) Appl. No.: 10/163,694

(22) Filed: Jun. 6, 2002

(51) Int. Cl. G02C 1/00; A61F 9/02

(52) U.S. Cl. 2/452; 351/43

(57) ABSTRACT

The present invention describes a mask or goggles having a strap which includes a hair restraint mechanism for maintaining the hair of the wearer away from the wearer's face and/or equipment, while the mask is in place. The strap includes a central body which fits around the back of the wearer's head, fastening straps extending from the central body to attach the strap to the mask body and a hair restraint portion extending from the central body. Typically, the hair restraint portion is perpendicular to the central body and extends downward therefrom. The hair restraint portion includes a mechanism for fastening or securing it around the wearer's hair during use.

### BACKGROUND OF THE INVENTION

1. Field of the Invention

The present invention relates generally to the field of retaining devices for masks. Specifically, the present invention is a strap for a mask, preferably a scuba mask, having a hair retention apparatus included therein.

2. Discussion of the Prior Art

Most masks, particularly those used for scuba diving, snorkeling and the like, include a mask portion, having a clear, typically glass, panel and a seal to prevent water from coming between the wearers face and the glass. A strap is typically included that extends from one side of the mask portion, around the back of the wearer's head and attaches at the opposite side of the mask portion. The strap maintains a certain amount of tension to adequately pull the seal against the wearer's face to form a water-tight seal.

In certain circumstances, particularly in the case of scuba diving and snorkeling, those having long hair can experience a number of difficulties in managing their hair. For instance, once in the water and especially once underwater, long hair tends to “free float” due to the relatively light weight of the hair. This can cause the hair to become entangled in equipment, obscure the vision of the diver or snorkeler, interfere with the seal between the mask and the wearer's face or simply become knotted or entangled in itself, thereby requiring significant time and effort to untangle.

There are a number of methods or devices for restraining hair. Various styles of elastic bands are commonly used, as are various styles of plastic or metal pins, clasps or clamps. These implements can cause significant problems, however, when worn with a mask. The strap of the mask typically runs around the back of the wearer's head. This is the same area that bands, clasps and the like are typically worn. The tension of the mask strap pulling a hair restraint device against the wearer's head can cause significant discomfort. So much so, the most of the time, it is preferable to simply allow the hair to go unrestrained. In addition, these devices are easily lost or forgotten, thereby leaving the wearer with no means of hair control.

Another method commonly used for restraining hair is braiding or weaving. This is a popular alternative to the use of the hair restraint devices described above because is does not cause as much discomfort when a mask is worn. This method, however, can be expensive and time consuming.

Therefore, it would be desirable to have an apparatus that was comfortable and convenient for hair restraint in conjunction with the wearing of a mask.

### SUMMARY OF THE INVENTION

The present invention is a strap for a mask having an apparatus or mechanism for controlling the wearer's hair. The strap is attached to the mask using any suitable means and extends across the back of the wearer's head, thereby securing the mask to the wearer's face. The strap further includes a hair retention or restraint mechanism for maintaining the users hair in a secured fashion while the mask is in place. In this way, the hair will not become entangled or otherwise interfere with the wearer's movement, vision or operation of other equipment the wearer may be operating.

In a preferred embodiment, the strap is used for retain a scuba mask securely about the wearer's face, while also preventing the wearer's hair from becoming tangled or knotted in itself or in the various hoses and other equipment typically worn by scuba divers. The strap will also prevent the wearer's hair from obscuring the wearer's vision, particularly while the wearer is underwater.

The strap may be affixed, using suitable connectors or other retaining devices, to a mask for retaining the mask securely on the wearers face. Preferably, the strap provides an adjustment mechanism for allowing the wearer to tighten or loosen the strap as required for optimum fit and comfort. The strap includes a retaining portion which is typically positioned around the wearer's head, provides tension to retain the mask in position about the wearer's face. The strap further includes a hair retaining or hair restraint portion which may be secured around the wearer's hair. The hair restraint portion is attached to the retaining portion and is typically positioned perpendicular to the retaining portion, extending downward therefrom. Any suitable attachment method may be used to secure the hair restraint portion to the retaining portion, such as, by way of example, sewing, adhesive or heat-based methods. Alternatively, the two portions may be prepared or produced from a single piece of material. The hair restraint portion may further include a mechanism or device for securing around the hair of the wearer. The strap may be formed from any suitable material. However, in the case of a scuba mask, the material should be capable of withstanding immersion in water.

In a preferred embodiment, the strap includes a central retaining body for fitting around the back of the wearer's head. Right and left mask attachment straps are affixed or secured to the central body and interface through a suitable latching or connecting mechanism to the mask body. The hair retaining body extends substantially downward from the central body and preferably includes a hair restraint mechanism.

In an alternative embodiment, the strap may be adapted for use with swimming goggles, sport goggles or the like. The strap may be used in association with skiing goggles or protective eyewear, as well. A central retaining body is included having a hair retaining portion extending therefrom. Right and left attachment straps may be included for attachment to the goggles or eyewear or slits or aperatures may be placed in the central portion to allow an existing strap to pass through and around the central portion, thereby allowing the device to be retrofit to an existing pair of goggles.

In yet another embodiment, the strap may be included with or incorporated into protective eyewear, such as that worn in an industrial setting or any other environment in which it would be beneficial to restrain or control the wearer's hair. There are numerous safety concerns when individuals with longer hair work around equipment, particularly, for instance, rotating equipment. The present invention is useful in preventing the wearer's hair from becoming entangled in such equipment, thereby preventing the possilibity of serious or significant injury.

### BRIEF DESCRIPTION OF THE DRAWINGS

FIG. 1 is a schematic showing a mask having a mask strap in accordance with the present invention.

FIG. 2 is a schematic of a first mask strap in accordance with the present invention.

FIG. 3 is a schematic showing a second mask strap in accordance with the present invention.

FIG. 4 is a schematic showing the hair restraint portion.

FIG. 5 is a schematic showing an alternate embodiment of the hair restraint portion.

FIG. 6 is a schematic showing a pair of goggles incorporation the present invention.

### DETAILED DESCRIPTION OF THE PREFERRED EMBODIMENTS

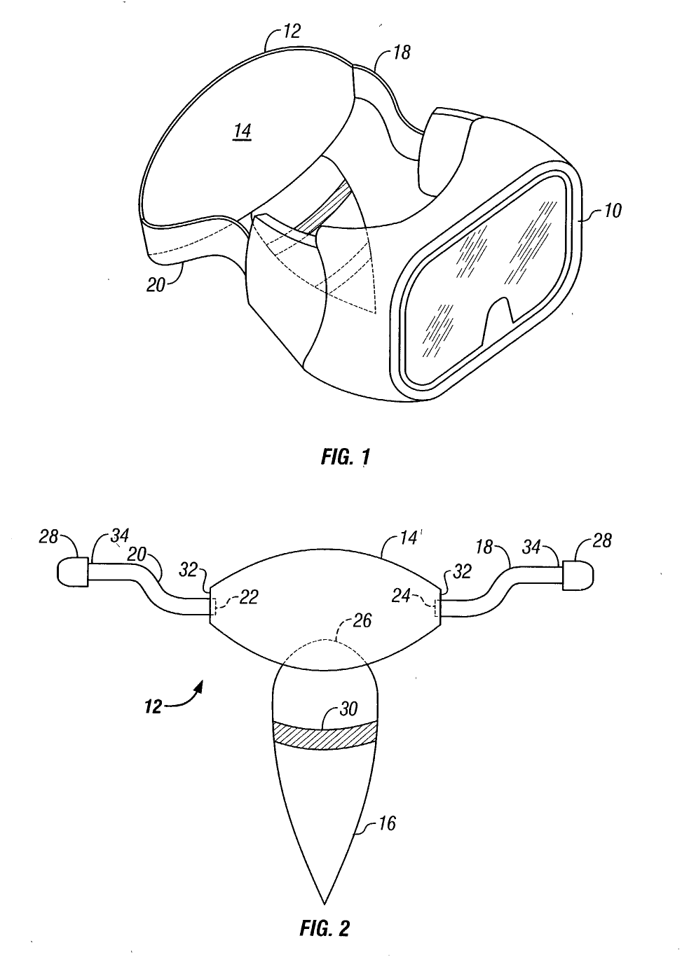

FIG. 1 shows a mask 10 having a strap 12 in accordance with the present invention. FIG. 2 shows the strap 12 having a central body or retaining portion 14 which fits substantially around the back of the wearer's head when the mask is in use. The central body 14 has mounting or fastening straps 18, 20 attached or secured to ends 32. The fastening straps may be attached by any suitable method, such as sewing, adhesive or heat-based methods. Alternatively, the fastening straps 18, 20 may be integrated into the central body 14, thereby alleviating the need for attachment to the central body 14. At each free end 34, that is, the end that is not attached to the central body, each fastening strap 18, 20 has a latch or securing mechanism 28 for securing the straps to the mask body 11. Any suitable mechanism may be used to secure the straps to the mask body. In a preferred embodiment, the straps are attached to the mask body with a suitable clasp, clamp or other device and may be adjusted such that the tension created by the strap is sufficient to allow the mask body to create a water tight seal around the wearer's face, without being uncomfortable.

Extending from the central body is a hair restraint portion 16 of the strap 12. The hair restraint portion may include any suitable attachment method or mechanism 26. In one embodiment, the hair restraint portion is sewn to the central body. In another embodiment, the hair restraint portion may be affixed to the central body using a waterproof adhesive. The hair restraint portion 16 includes a fastening or joining device 30 to allow the hair restraint portion to enclose or surround the wearer's hair, thereby restraining the hair in a secure and unobtrusive position. Any suitable fastening device may be used. For instance, snaps, buttons or hooks could all be used in the present invention. In a preferred embodiment, the fastening device is a hook and loop material, such as Velcro® the extends laterally across the hair restraint portion to all said portion to be substantially rolled around the wearer's hair, thereby forming a tube-type enclosure for the hair. Alternatively, a single or a plurality of straps may be fastened to the hair restraint portion, wherein the straps may be permanently secured at one end and removeably secured at a second end, thereby allowing the strap to be positioned substantially around the wearer's hair and then fastened to the hair restraint portion, thereby retaining the wearer's hair during use.

The strap of the present invention may be formed from any suitable material. Preferably the material used will be water-resistant or waterproof. In addition, it is preferable to use a material that is resilient or elastic. It is further preferred to use a material that is buoyant. Neoprene is a preferred material, particularly for both the central portion and the hair restraint portion. It may be preferable to form the fastening straps from a less elastic or substantially non-elastic material, such as nylon, polypropylene or the like.

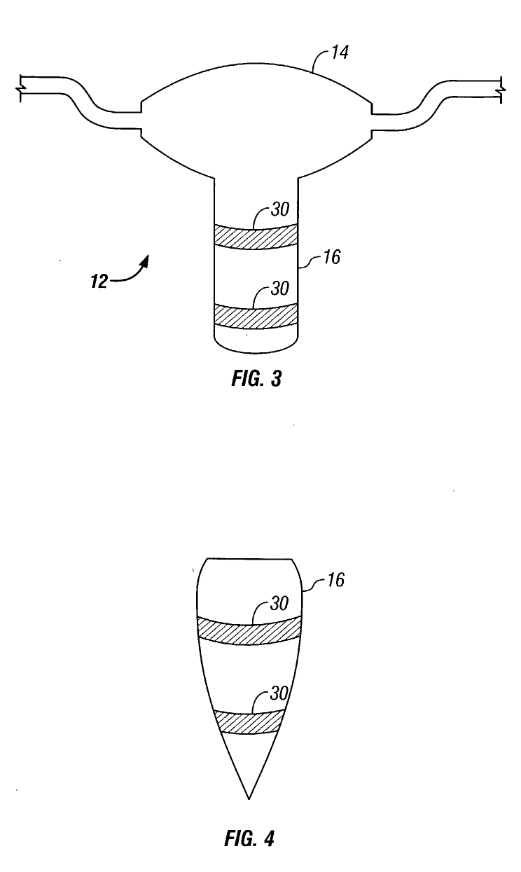

FIG. 3 shows yet another preferred embodiment of the present invention. The central portion 14 is formed from a single piece or as a single unit in conjunction with the hair restrain portion 16. In this way, the relative disadvantages encountered when using a two-piece design may be eliminated. For instance, separation of the seam or connecting mechanism is no longer possible. In addition, certain advantages in terms of production cost and time may be realized by forming the central portion and hair restraint portion as a single unit. Any suitable fastening or joining device or mechanism 30 may be employed, as previously described and as further described, below.

FIG. 4 shows an isolated schematic of the hair restraint portion 16 of the strap 12. As shown, the hair restraint portion is substantially V-shaped. Disposed on the hair restraint portion is a fastening or joining device or mechanism 30. It should be understood that the hair restraint portion may be formed in any suitable shape or configuration that allows the wearer's hair to be adequately restrained to prevent entanglement or interference with the wearer or equipment. The length of the hair restraint portion need only be sufficient to maintain the wearer's hair in a position or location such that the likelihood or interference with either the wearer or her equipment is reduced. In certain instances or situations, a longer hair restraint portion may be beneficial or desirable to prevent the wearer's hair from becoming entangled in itself. This may particularly be the case where the wearer's hair is relatively long.

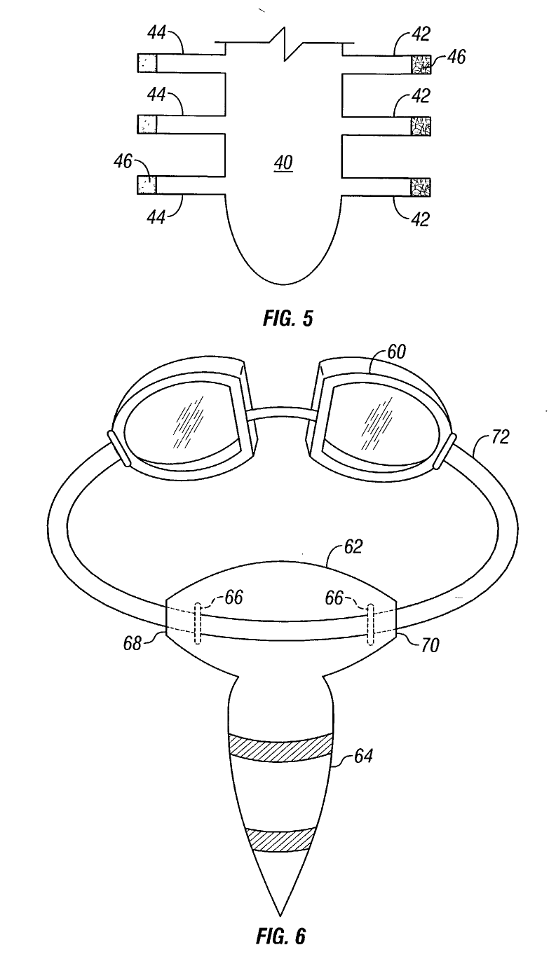

In one embodiment, a substantially rectangular portion may be used to form the hair restraint portion. In an alternative embodiment and as shown in FIG. 5 a configuration having a central backbone 40 with a plurality of straps or ties 42 extending therefrom may also be used. Each strap 42 will preferably have an opposing counterpart 44 and each strap/counterpart combination may have suitable a suitable fastening mechanism 46 for joining each strap to it's respective counterpart. The central backbone 40 may be secured or affixed in any suitable position relative to the central body 14, but is preferably positioned medially and perpendicular to said body. In use, each strap/counterpart combination may be wrapped or enclosed around the wearer's hair to retain the hair while the mask is in use.

In yet another embodiment and as shown in FIG. 6, the present invention may be incorporated with, attached to or retrofit to a pair of goggles 60 or protective eyewear. The goggles may be any suitable type, but the device is particularly suited for use with swimming or “sport-type” goggles, such as those used for skiing. The device may also be incorporated or used with protective eyewear, such as goggles worn for eye protection in an industrial or “shop” environment. In this capacity, the device may provide both convenience restraining or managing the wearer's hair and may also provide significant safety benefits by preventing the wearer's hair from becoming entangled in equipment. More preferably, the goggles or eyewear will incorporate a strap for retaining the eyewear on the wearer's head.

The device has a central body 62 and a hair restraint portion 64. In one embodiment, the central body has a pair of slits 66 positioned near the left and right edges 68, 70, respectively, so that an existing goggle strap 72 may pass through the slits thereby securing the device to the goggles. Alternatively, the device may provide a pair of fastening straps for interfacing and securing the strap to the goggles. Regardless of the mounting or attachment mechanism used, the device may be used to control or restain the wearer's hair.

I claim:

1. A hair restraint strap comprising:
(a) a lateral retaining portion having a left end and a right end;
(b) a hair restraint portion extending from said lateral portion;
(c) right and left coupling members attached to said right and left ends, respectively.

2. The strap of claim 1 wherein the retaining portion is buoyant.

3. The strap of claim 2, wherein said retaining portion is neoprene.

4. The strap of claim 1, wherein said coupling members are substantially non-elastic.

5. The strap of claim 4, wherein said coupling members are nylon.

6. The strap of claim 1, wherein said hair restraint portion has a convex V-shape.

7. The strap of claim 1, wherein said hair restraint portion further includes a fastening mechanism.

8. The strap of claim 7, wherein said fastening mechanism is a hook and loop material.

9. The strap of claim 1, wherein said hair restraint portion comprises a central backbone having a plurality of straps extending therefrom.

10. The strap of claim 1, further comprising goggles.

11. The strap of claim 1, further comprising protective eyewear.

12. A mask having a strap, wherein said strap comprises:
(a) a lateral mask retaining portion having a left end and a right end;
(b) a hair restraint portion extending from said lateral portion;
(c) right and left coupling members attached to said right and left ends, respectively, for securing said strap to a mask.

13. A hair restraint apparatus comprising:
(a) a lateral retaining portion having a left end and a right end; and
(b) a hair restraint portion extending from said lateral portion,
wherein said lateral retaining portion has a left aperature formed near said left end and a right aperature formed near said right end.

14. The strap of claim 13, further comprising a pair of goggles.

15. The strap of claim 14, wherein said goggles comprise a strap for passing through the aperatures in said retaining portion.

16. The strap of claim 13, further wherein said strap is attached to protective eyewear.
